(19) United States
(12) Patent Application Publication
(10) Pub. No.: US 2025/0276051 A1
(43) Pub. Date: Sep. 4, 2025

(54) POWASSAN VIRAL ANTIGENS AND RELATED COMPOSITIONS, AND USES THEREOF TO VACCINATE AND TREAT PATIENTS

(71) Applicant: GeneOne Life Science, Inc., Seoul (KR)

(72) Inventors: Joel Maslow, Seoul (KR); Moonsup Jeong, Seoul (KR); Christine Roberts, Seoul (KR); Youngran Cho, Seoul (KR); Young Park, Seoul (KR)

(73) Assignee: GeneOne Life Science, Inc., Seoul (KR)

(21) Appl. No.: 17/786,310

(22) PCT Filed: Dec. 18, 2020
(86) PCT No.: PCT/US2020/066233

§ 371 Date: Jun. 16, 2022

Related U.S. Application Data
(60) Provisional application No. 62/949,821, filed on Dec. 18, 2019.

(51) Int. Cl. A61K 39/12; A61K 39/00; A61P 31/14; C07K 14/005; C12N 7/00

(52) U.S. Cl. CPC A61K 39/12; A61P 31/14; C07K 14/005; C12N 7/00; A61K 2039/53; A61K 2039/54; A61K 2039/575; C12N 2770/24122; C12N 2770/24134

(57) ABSTRACT

Provided herein is an immunogenic composition comprising a synthetic consensus antigen to POWV. Also disclosed herein is a method of treating an infection by POWV in a subject in need thereof, by administering the immunogenic composition to the subject.

### TECHNICAL FIELD

The present invention relates to compositions comprising an immunological Powassan virus antigen, compositions comprising a recombinant nucleic acid sequence for generating one or more synthetic antigens, and functional fragments thereof, and combinations thereof. The compositions of the invention provide improved methods for inducing immune responses, and for prophylactically and/or therapeutically immunizing individuals against Powassan virus.

### BACKGROUND

Vaccines help the body fight disease by training the immune system to recognize and destroy harmful substances and diseased cells. Vaccines can be largely grouped into two types, preventive and treatment vaccines. Preventive vaccines are given to healthy people to prevent the development of specific diseases, while treatment vaccines, also referred to as immunotherapies, are given to a person who has been diagnosed with disease.

Nucleic acid based vaccines work by inducing expression of a small fraction of genes within the host's cells, which in turn. enhance the ability of the host's immune system to eliminate the infecting pathogen. As such, clinical response of a viral vaccine can depend on the ability of the vaccine to obtain a high-level of immunogenicity and have sustained long-term expression.

The Powassan virus (POWV) is a flavivirus. The Powassan virus has been noted as the only tick-borne flavivirus in North America with human pathogenicity. Powassan virus is also found in the warm climate across Eurasia, where it is part of the tick-borne encephalitis virus-complex. Powassan encephalitis is severe, and neurologie sequelae are common. There are currently no medications or vaccines to treat or prevent the POWV. People affected by Powassan virus generally show symptoms 1 to 3 weeks after infection. The initial symptoms include fever, headache, nausea, occasional confusion, and weakness. Neurologic complications of Powassan virus infection may include: seizures, aphasia, cranial nerve palsies, paresis and altered mental status. About 10% of POWV encephalitis cases are fatal and half the survivors have permanent symptoms that affect their brain.

Therefore, there remains a need to discover novel compositions and methods to prevent POWV infection and for those affected, novel compositions and methods to treat POWV infections.

### SUMMARY

Aspects of the present invention are directed to immunological compositions comprising a nucleotide sequence encoding a consensus POWV antigen described herein. The consensus POWV antigen comprises an amino acid sequence that is prME, and can include a signal sequence such as JEV signal, prM signal or a portion of prM signal. The nucleotide sequence can include a leader sequence such as an IgE leader.

Some aspects of the present invention are directed to methods of treating or preventing POWV infection in a subject in need thereof by administering the immunological compositions provided herein.

### BRIEF DESCRIPTION OF DRAWINGS

FIG. 1 shows an image of a western blot of POWV-prM protein expression from transfected cells.

FIG. 2 shows graphs of ELISA analysis that illustrate measurement of antibody binding against POWV prME antigen from sera from the various construct (pVAX1, pVAX1-IgE leader-prME, pVAX1-IgE leader-portion of prM signal-prME, pVAX1-prM signal-prME, or pVAX1-JEV signal-prME) immunized mice.

FIG. 3 shows graphs of ELISA analysis that compare antibody binding against POWV prME between the various mouse sera (pVAX1, pVAX1-IgE leader-prME, pVAX1-IgE leader-portion of prM signal-prME, pVAX1-prM signal-prME, or pVAX1-JEV signal-prME transfected mice).

FIG. 4 shows graphs that provide endpoint titers for POWV-E specific antibodies for each of the constructs ((pVAX1, pVAX1-IgE leader-prME, pVAX1-IgE leader-portion of prM signal-prME, pVAX1-prM signal-prME, or pVAX1-JEV signal-prME).

### DETAILED DESCRIPTION OF EMBODIMENTS

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the immunogenic composition described herein to enhance the immunogenicity of the antigen.

“Antigen” as used herein means molecules that can be recognized by the immune system, and more specifically are bound by antibodies or a cell surface receptor on immune cells such as T-cells. Preferably, Antigens invoke an immune response, such as inducing a humoral or cell-mediated immune response. Antigens are usually proteins, peptides (amino acid chains) and polysaccharides (chains of monosaccharides/simple sugars) but lipids and nucleic acids become antigens only when combined with proteins and polysaccharides. The antigen may originate from within the body (“self-antigen”) or from the external environment (“non-self”).

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a nucleic acid sequence, or corresponding polypeptide sequence, constructed based on analysis of an alignment of multiple subtypes of a particular antigen. The sequence may be used to induce broad immunity against multiple subtypes, serotypes, or strains of a particular pathogen.

As used herein, the term “expressible form” refers to gene constructs that contain the necessary regulatory elements operably linked to a coding sequence that encodes a target protein, such that when present in the cell of the individual, the coding sequence will be expressed.

“Fragment” as used herein means a nucleotide sequence or a portion thereof that encodes a polypeptide capable of eliciting an immune response in a mammal. The fragments can be DNA or RNA fragments selected from at least one of the various nucleotide sequences that encode protein fragments set forth below.

“Immunogenic fragment” with respect to polypeptide sequences means a polypeptide capable of eliciting an immune response in a mammal that cross reacts with a full length endogenous antigen. Fragments of consensus proteins can comprise at least 80%, at least 90% or at least 95% of a consensus protein. In some embodiments, fragments of consensus proteins can comprise at least 520 amino acids or more, at least 530 amino acids or more, at least 540 amino acids or more, at least 550 amino acids or more, at least 560 amino acids or more, at least 570 amino acids or more, at least 580 amino acids or more, at least 590 amino acids or more, at least 600 amino acids or more, at least 610 amino acids or more, at least 620 amino acids or more, at least 630 amino acids or more, at least 640 amino acids or more, at least 650 amino acids or more, or at least 660 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a tumor microenvironment protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that is capable of being administered the immunogenic compositions described herein. The mammal can be, for example, a human, chimpanzee, dog, cat, horse, cow, rabbit, groundhog, squirrel, mouse, rat, or other rodents.

“Substantially identical” as used herein can mean that a first and second amino acid sequence are at least 80%, 81%, 82%, 83%, 84%, 85%, 86%, 87%, 88%, 89%, 90%, 91%, 92%, 93%, 94%, 95%, 96%, 97%, 98%, or 99% over a region of 20, 21, 22, 23, 24, 25, 30, 35, 40, 45, 50, 55, 60, 65, 70, 75, 80, 85, 90, 95, 100, 200, 300, 400, 500, 600 or more amino acids. Substantially identical can also mean that a first nucleotide sequence and a second nucleotide sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of a subject from a disease through means of preventing, suppressing, repressing, or completely eliminating the disease. In one embodiment, preventing the disease involves administering an immunogenic composition of the present invention to a subject prior to onset of the disease. In one embodiment, preventing the disease involves administering an immunogenic composition of the present invention to a subject following a treatment so as to prevent reoccurrence or further progression of the disease. Suppressing the disease involves administering an immunogenic composition of the present invention to a subject after induction of the disease but before its clinical appearance. Repressing the disease involves administering an immunogenic composition of the present invention to a subject after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleotide sequence that is substantially identical over the full length of the full gene sequence or a fragment thereof. The nucleotide sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, adeno-associated virus (AAV), bacterial artificial chromosome or yeast artificial chromosome. A vector can be a DNA or RNA vector. A vector can be an extrachromosomal vector, and preferably, is a DNA plasmid or mRNA.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Description

Aspects of the present invention are directed to immunological compositions comprising a nucleotide sequence encoding a consensus POWV antigen described herein. The consensus POWV antigen comprises an amino acid sequence that is prME, and can include a signal sequence such as JEV signal, prM signal or a portion of prM signal. The nucleotide sequence can include a leader sequence such as an IgE leader.

Some aspects of the present invention are directed to methods of treating or preventing POWV infection in a subject in need thereof by administering the immunological compositions provided herein.

In one embodiment, the POWV antigen comprises an antigen selected from the group consisting of a POWV-prME. In some embodiments, there are nucleotide expression constructs comprising prME plus an N-terminal signal sequence, which can be a JEV signal or a prM signal, or fragments thereof. In other embodiments, one or more of the aforementioned nucleotides is also comprised of a N-terminal leader sequence, which in some embodiments can be an IgE leader sequence.

The present disclosure provides an optimized consensus sequence encoding a POWV antigen. In one embodiment, the POWV antigen encoded by the optimized consensus sequence is capable of eliciting an immune response in a mammal. In one embodiment, the POWV antigen encoded by the optimized consensus sequence can comprise an epitope(s) that makes it particularly effective as an immunogen against which an immune response can be induced.

The consensus sequence can be derived from two or more native POWV nucleic acid sequences. Modifications to the consensus sequence can include codon optimization, RNA optimization, addition of a Kozak sequence for increased translation initiation, and/or the addition of an immunoglobulin or other leader sequence to increase immunogenicity. The encoded POWV antigen can comprise a signal peptide such as an immunoglobulin signal peptide, for example, but not limited to, an immunoglobulin E (IgE) or immunoglobulin (IgG) signal peptide. The encoded antigen by the POWV sequence can be designed to elicit stronger cellular and/or humoral immune responses than a corresponding native antigen.

In one embodiment, the encoded POWV antigen is operably linked to one or more regulatory elements. In one embodiment, a regulatory element is a leader sequence. In one embodiment, the POWVDNA sequence operably linked to an IgE leader encoding sequence is set forth in SEQ ID NO:6. In one embodiment, the optimized consensus-encoded POWV antigen operably linked to an IgE leader sequence.

In some embodiment, the POWV antigen is an immunogenic domain of one or more of the functional proteins In some instances, the POWV antigen is the D3 domain of the envelope protein (E). In some instances, the nucleotide constructs of the invention include the D3 domain of the E protein repeated in the open reading frame, and in particular repeated three times, thereby expressing three D3 domains.

In one embodiment, a regulatory element is a start codon. Therefore, in one embodiment, the invention relates to a nucleic acid sequence as set forth in SEQ ID NO:1 (pVAX1-IgE leader-prME), SEQ ID NO:2 (pVAX1-IgE leader-prMsignal-prME), SEQ ID NO:3 (pVAX1-IgE leader-portion of prMsignal-prME), SEQ ID NO:4 (pVAX1-prM signal-prME), or SEQ ID NO:5 (pVAX1-JEV signal-prME). In one embodiment, the invention relates to an amino acid sequence as set forth in SEQ ID NO:7 prM consensus or SEQ ID NO:8 E consensus, or a fragment or homolog thereof, operably linked to an amino acid encoded by a start codon (e.g., a Methionine) at the N-terminus.

In one embodiment, a regulatory element is at least one stop codon. Therefore, in one embodiment, the invention relates to a nucleic acid sequence as set forth in SEQ ID NO:9 (consensus prME) or a fragment or homolog thereof, operably linked to a nucleotide sequence comprising at least one stop codon at the 3′ terminus. In one embodiment, the nucleotide sequence is operably linked to two stop codons to increase the efficiency of translational termination.

In one embodiment, the optimized consensus sequence encoding a POWV antigen can encode a peptide having the amino acid sequence set forth in SEQ ID NO:7 or SEQ ID NO: 8. In one embodiment, the optimized consensus sequence can have the nucleotide sequence set forth in SED ID NO: 9, which is a part of the inserts in constructs SEQ ID NO: 1, 2, 3, 4, or 5. In some embodiments, the sequence can be the nucleotide sequence having at least about 96%, 97%, 98%, 99% or 100% identity over an entire length of the nucleotide sequence set forth in SEQ ID NO:9. In other embodiments, sequence can be the nucleotide sequence that encodes the amino acid sequence having at least about 96%, 97%, 98%, 99%, or 100% identity over an entire length of the amino acid sequence set forth in SEQ ID NO:7 or SEQ ID NO:8. In some embodiments, the optimized consensus POWV antigen can be encoded by an RNA that is a transcript from a DNA sequence having at least about 96%, 97%, 98%, 99% or 100% identity over an entire length of the nucleic acid sequence set forth in the SEQ ID NO:9. In some embodiments, the optimized consensus POWV antigen can be encoded by an RNA that encodes an amino acid sequence having at least about 96%, 97%, 98%, 99% or 100% identity over an entire length of the amino acid sequence set forth in SEQ ID NO:7 or SEQ ID NO:8.

In some embodiments, the antigen can have an amino acid sequence having at least about 96%, 97%, 98%, 99%, or 100% identity over an entire length of the amino acid sequence set forth in SEQ ID NO:7 or SEQ ID NO:8.

Immunogenic fragments of SEQ ID NO:7 or SEQ ID NO:8 can be provided. Immunogenic fragments can comprise at least at least 80%, at least 85%, at least 90%, at least 95%, at least 96%, at least 97%, at least 98% or at least 99% of the full length of SEQ ID NO:7 or SEQ ID NO:7. In some embodiments, immunogenic fragments include a leader sequence, such as for example an immunoglobulin leader, such as the IgE leader. In some embodiments, immunogenic fragments are free of a leader sequence.

In one embodiment, the nucleic acid sequence comprises an RNA sequence encoding a consensus POWV immunogen sequence described herein. For example, nucleic acids may comprise an RNA sequence encoding one or more of SEQ ID NO:7 or SEQ ID NO:8, a variant thereof, a fragment thereof or any combination thereof.

Immunogenic fragments of proteins with amino acid sequences homologous to immunogenic fragments of SEQ ID NO:7 or SEQ ID NO:8 can be provided. Such immunogenic fragments can comprise at least 80%, at least 85%, at least 90%, at least 95%, at least 96%, at least 97%, at least 98% or at least 99% of proteins that are 95% homologous to SEQ ID NO:7 or SEQ ID NO:8. Some embodiments relate to immunogenic fragments that have 90% homology to the immunogenic fragments of consensus protein sequences herein. Some embodiments relate to immunogenic fragments that have 97% homology to the immunogenic fragments of consensus protein sequences herein. Some embodiments relate to immunogenic fragments that have 98% homology to the immunogenic fragments of consensus protein sequences herein. Some embodiments relate to immunogenic fragments that have 99% homology to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:9. Immunogenic fragments can be at least 80%, at least 85%, at least 90%, at least 95%, at least 96%, at least 97%, at least 98% or at least 99% of the full length of SEQ ID NO:9. Immunogenic fragments can be at least 96%, at least 97% at least 98% or at least 99% homologous to fragments of SEQ ID NO:9. In some embodiments, immunogenic fragments include sequences that encode a leader sequence, such as for example an immunoglobulin leader, such as the IgE leader. In some embodiments, fragments are free of coding sequences that encode a leader sequence.

### Immunogenic Composition

Provided herein are immunogenic compositions, such as vaccines, comprising an antigenic sequence, an encoded antigen, a fragment thereof, a variant thereof, or a combination thereof.

The immunogenic composition can be a DNA vaccine, an RNA vaccine, a peptide vaccine, or a combination vaccine. The vaccine can include an optimized consensus nucleotide sequence encoding an antigen. The nucleotide sequence can be DNA, RNA, mRNA, cDNA, a variant thereof, a fragment thereof, or a combination thereof. The nucleotide sequence can also include additional sequences that encode linker, leader, or tag sequences that are linked to the antigen by a peptide bond. The peptide vaccine can include an antigen, a variant thereof, a fragment thereof, or a combination thereof. The combination DNA and peptide vaccine can include the above described optimized consensus nucleotide sequence and the encoded antigen.

The vaccine of the present invention can have features required of effective vaccines such as being safe so that the vaccine itself does not cause illness or death; being protective against illness; inducing neutralizing antibody; inducing protective T cell responses; and providing ease of administration, few side effects, and biological stability.

### Immune Response

The immunogenic composition can induce an immune response in the subject administered the composition.

The immunogenic composition can further induce an immune response when administered to different tissues such as the muscle or skin. The immunogenic composition can further induce an immune response when administered via electroporation, or injection, or subcutaneously, or intramuscularly.

### Fragments

In one embodiment, the immunogenic fragment is an immunogenic fragment of a full length antigen of the invention. As used herein, an immunogenic fragment is a fragment of a full length nucleic acid or amino acid sequence that can induce an immune response significantly similar to that of the full length sequence. In one embodiment, an immunogenic fragment comprises an immunogenic epitope of a full length sequence. In one embodiment, the immunogenic fragment induces an immune response at least about 0.7-fold, at least about 0.8-fold, at least about 0.9-fold, at least about 1.0-fold, at least about 1.1-fold, at least about 1.2-fold, at least about 1.3-fold, at least about 1.4-fold, at least about 1.5-fold, at least about 2.0-fold or greater than 2.0-fold as compared to the full length sequence.

The immunogenic fragment can induce a humoral immune response in the subject administered the immunogenic fragment. The humoral immune response can be induced in the subject administered the immunogenic fragment by about 1.5-fold to about 16-fold, about 2-fold to about 12-fold, or about 3-fold to about 10-fold. The humoral immune response can be induced in the subject administered the immunogenic fragment by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered immunogenic fragment.

The humoral immune response induced by the immunogenic fragment can include an increased level of IgG antibodies associated with the subject administered the immunogenic fragment as compared to a subject not administered the immunogenic fragment The level of IgG antibody associated with the subject administered the immunogenic fragment can be increased by about 1.5-fold to about 16-fold, about 2-fold to about 12-fold, or about 3-fold to about 10-fold as compared to the subject not administered the immunogenic fragment. The level of IgG antibody associated with the subject administered the immunogenic fragment can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to a subject not administered the immunogenic fragment.

The induced cellular immune response can include an increased CD8⁺ T cell response associated with the subject administered the immunogenic fragment as compared to the subject not administered the immunogenic fragment. The CD8⁺ T cell response associated with the subject administered the immunogenic fragment can be increased by about 2-fold to about 30-fold, about 3-fold to about 25-fold, or about 4-fold to about 20-fold as compared to the subject not administered the immunogenic fragment. The CD8⁺ T cell response associated with the subject administered the immunogenic fragment can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23 0-fold, at least about 24.0-fold, at least about 25.0-fold, at least about 26.0-fold, at least about 27.0-fold, at least about 28.0-fold, at least about 29.0-fold, or at least about 30.0-fold as compared to a subject not administered the immunogenic fragment.

The induced cellular immune response can include an increased frequency of CD107a/IFNγ/T-bet triple-positive CD8 T cells that are reactive against the native antigen. The frequency of CD107a/IFNγ/T-bet triple-positive CD8 T cells associated with the subject administered the immunogenic fragment can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not administered the immunogenic fragment.

The induced cellular immune response can include an increased frequency of CD107a/IFNγ double-positive CD8 T cells that are reactive against the native antigen. The frequency of CD107a/IFNγ double-positive CD8 T cells associated with the subject administered the immunogenic fragment can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to a subject not administered the immunogenic.

The cellular immune response induced by the immunogenic fragment can include eliciting a CD4⁺ T cell response. The elicited CD4⁺ T cell response can be reactive with the native antigen genetically related to the optimized consensus antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD4⁺ T cells that produce IFN-γ. The frequency of CD4 IFN-γ⁺ T cells associated with the subject administered the immunogenic fragment can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to a subject not administered the immunogenic fragment.

The induced cellular immune response can include an increased frequency of CD4⁺ T cells that produce TNF-α. The frequency of CD4⁺ TNF-α⁺ T cells associated with the subject administered the immunogenic fragment can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to a subject not administered the immunogenic fragment.

The induced cellular immune response can include an increased frequency of CD4⁺ T cells that produce both IFN-γ and TNF-α. The frequency of CD4⁺IFN-γ⁺TNF-α⁺ associated with the subject administered the immunogenic fragment can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to a subject not administered the immunogenic fragment.

The immunogenic fragment of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The immunogenic fragment can further induce an immune response when administered to different tissues such as the muscle or skin. The immunogenic fragment can further induce an immune response when administered via electroporation, or injection, or subcutaneously, or intramuscularly.

### Vector

The nucleotide construct described above can be placed in one or more vectors. The one or more vectors can contain an origin of replication. The one or more vectors can be a plasmid, RNA, viral, including vesicular stomatitis virus, measles virus, modified vaccinia Ankara virus, adenovirus, AAV, bacteriophage, bacterial artificial chromosome or yeast artificial chromosome. The one or more vectors can be either a self-replication extra chromosomal vector.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” that may be extra-chromosomal circular or linear DNA. Where a vector is being maintained by a host cell, the vector may either be stably replicated by the cells during mitosis as an autonomous structure, or is incorporated within the host's genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector.

The vectors may have expression signals such as a strong promoter, a strong termination codon, adjustment of the distance between the promoter and the cloned gene, and the insertion of a transcription termination sequence and a PTIS (portable translation initiation sequence).

### Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### Plasmid

The one or more vectors can be a plasmid. The plasmid may be useful for transfecting cells with the recombinant nucleic acid construct. The plasmid may be useful for introducing the recombinant nucleic acid construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid can also be pIDV or pIDV-II.

The plasmid may be pSE420 (Invitrogen, San Diego, Calif.), which may be used for protein production in Escherichia coli (E.coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90% homologous to SEQ ID NO:9, or a variant thereof or a fragment thereof. In another embodiment, the nucleotide sequence comprises an RNA sequence transcribed by a DNA sequence encoding a polypeptide sequence at least 90% homologous to one of SEQ ID NO:7 or SEQ ID NO:8 or a variant thereof or a fragment thereof. Accordingly, in one embodiment, the invention provides an RNA molecule encoding one or more of the POWV antigens. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′. UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vectors

The vector may be a circular plasmid, which may transform a target cell and exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid immunogenic composition, or linear expression cassette (“LEC”), that is capable of being efficiently delivered to a subject and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleotide sequences unrelated to the desired antigen gene expression.

The LEC may be derived from any plasmid capable of being linearized. The plasmid may be capable of expressing the antigen. The plasmid can be pNP (Puerto Rico/34) or pM2 (New Caledonia/99). The plasmid may be pVAX, pVAX1 pcDNA3.0, pIDV, pIDV-II, or provax, or any other expression vector capable of expressing DNA encoding the antigen and enabling a cell to translate the sequence to an antigen that is recognized by the immune system.

The LEC can be pcrM2. The LEC can be perNP. pcrNP and pcrMR can be derived from pNP (Puerto Rico/34) and pM2 (New Caledonia/99), respectively.

### Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleotide sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, CAG promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, adenoviruses, adeno-associated viruses, measles virus, vesicular stomatitis virus, modified vaccinia Ankara virus. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

### Multiple Vectors

The immunogenic composition may comprise a plurality of copies of a single nucleic acid molecule such a single plasmid, or a plurality of copies of two or more different nucleic acid molecules such as two or more different plasmids. For example an immunogenic composition may comprise plurality of two, three, four, five, six, seven, eight, nine or ten or more different nucleic acid molecules. Such compositions may comprise plurality of two, three, four, five, six, or more different plasmids.

Immunogenic compositions may comprise nucleic acid molecules, such as plasmids, that collectively contain coding sequence for a POWV antigen. Immunogenic compositions may comprise nucleic acid molecules, such as plasmids, that collectively contain coding sequence for multiple antigens. In one embodiment, the antigens are a POWV antigen and one or more additional adjuvants. Immunogenic compositions may comprise nucleic acid molecules, such as plasmids, that collectively contain coding sequence for one or more antigen and one or more adjuvant.

### Methods

Provided herein are methods of treating, protecting against, and/or preventing a POWV associated disease in a subject in need thereof by administering one or more immunogenic composition described herein to the subject. Administration of the immunogenic composition to the subject can induce or elicit an immune response in the subject.

Provided herein is a method for delivering the immunogenic composition for providing genetic constructs and proteins of the consensus antigen which comprise epitopes that make them particular effective against POWV. The method of delivering the immunogenic composition or vaccination may be provided to induce a therapeutic and prophylactic immune response. The vaccination process may generate in the mammal an immune response against POWV. The immunogenic composition may be delivered to an individual to modulate the activity of the mammal's immune system and enhance the immune response. The delivery of the immunogenic composition may be the transfection of the consensus antigen as a nucleic acid molecule that is expressed in the cell and delivered to the surface of the cell upon which the immune system recognized and induces a cellular, humoral, or cellular and humoral response. The delivery of the immunogenic composition may be used to induce or elicit and immune response in mammals against POWV by administering to the mammals the immunogenic composition as discussed above.

Upon delivery of the immunogenic composition and plasmid into the cells of the mammal, the transfected cells will express and secrete consensus antigens for each of the plasmids injected from the immunogenic composition. These proteins will be recognized as foreign by the immune system and antibodies will be made against them. These antibodies will be maintained by the immune system and allow for an effective response.

The immunogenic composition may be administered to a mammal to elicit an immune response in a mammal. The mammal may be human, primate, non-human primate, cow, cattle, sheep, goat, antelope, water buffalo, bison, bovids, deer, hedgehogs, rabbits, mice, rats, groundhogs, squirrels, other rodents, and chicken.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced cellular immune response can include a CD8⁺ T cell response, which is induced by about 2-fold to about 30-fold, about 3-fold to about25-fold, or about 4-fold to about 20-fold.

The immunogenic composition dose can be between 1 μg to 10 mg active component/kg body weight/time, and can be 20 μg to 10 mg component/kg body weight/time. The immunogenic composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of immunogenic composition doses for effective treatment can be 1, 2, 3, 4, 5, 6, 7, 8, 9, or 10.

The immunogenic composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration.

The immunogenic composition can be administered prophylactically or therapeutically. In prophylactic administration, the immunogenic compositions can be administered in an amount sufficient to induce an immune response. In therapeutic applications, the immunogenic compositions are administered to a subject in need thereof in an amount sufficient to elicit a therapeutic effect. An amount adequate to accomplish this is defined as “therapeutically effective dose.” Amounts effective for this use will depend on, e.g., the particular composition of the immunogenic composition regimen administered, the manner of administration, the stage and severity of the disease, the general state of health of the subject, and the judgment of the prescribing physician.

The immunogenic composition can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997), and Carson et al. (U.S. Pat. No. 5,679,647, issued Oct. 21, 1997), the contents of all of which are incorporated herein by reference in their entirety. The nucleic acid of the immunogenic composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The immunogenic composition can be delivered via a variety of routes. Typical delivery routes include parenteral administration, e.g., intradermal, intraepidermal, intramuscular or subcutaneous delivery. Other routes include oral administration, intranasal, and intravaginal routes. For the nucleic acid of the immunogenic composition in particular, the immunogenic composition can be delivered to the interstitial spaces of tissues of an individual (Felgner et al., U.S. Pat. Nos. 5,580,859 and 5,703,055, the contents of all of which are incorporated herein by reference in their entirety). The immunogenic composition can also be administered to muscle, or can be administered via intradermal or subcutaneous injections, or transdermally, such as by iontophoresis. Epidermal administration of the immunogenic composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The immunogenic composition can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the immunogenic composition.

The immunogenic composition can be a liquid preparation such as a suspension, syrup or elixir. The immunogenic composition can also be a preparation for parenteral, subcutaneous, intradermal, intramuscular or intravenous administration (e.g., injectable administration), such as a sterile suspension or emulsion.

The immunogenic composition can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

### Combination Treatments

The immunogenic composition may be administered in combination with other proteins and/or genes encoding CCL20, α-interferon, γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15 including IL-15 having the signal sequence deleted and optionally including the different signal peptide such as the IgE signal peptide, MHC, CD80, CD86, IL-28, IL-1, IL-2, IL-4, IL-5, IL-6, IL-10, IL-18, MCP-1, MIP-la, MIP-1B, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1,DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFKB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or combinations thereof. In some embodiments, the immunogenic composition is administered in combination with one or more of the following nucleic acid molecules and/or proteins: nucleic acid molecules selected from the group consisting of nucleic acid molecules comprising coding sequence that encode one or more of CCL20, IL-12, IL-15, IL-28, CTACK, TECK, MEC and RANTES or functional fragments thereof, and proteins selected from the group consisting of: CCL02, IL-12 protein, IL-15 protein, IL-28 protein, CTACK protein, TECK protein, MEC protein or RANTES protein or functional fragments thereof.

### Routes of Administration

The vaccine or pharmaceutical composition can be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, intradermal, intraepidermal, and intraarticular or combinations thereof. For veterinary use, the composition can be administered as a suitably acceptable formulation in accordance with normal veterinary practice. The veterinarian can readily determine the dosing regimen and route of administration that is most appropriate for a particular animal. The vaccine can be administered by traditional syringes, needleless injection devices, microneedle device, “microprojectile bombardment gene guns”, or other physical methods such as electroporation (“EP”), “hydrodynamic method”, or ultrasound.

The vector of the vaccine can be administering to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The one or more POWV constructs can be administered via DNA injection, and can optionally be accompanied by methods to enhance cellular uptake that may include magnetoporation, ultrasound, heat, electroporation, physical methods such as tattooing.

The immunogenic composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof.

The immunogenic composition may be administered by traditional syringes, needleless injection devices, “microprojectile bombardment gene guns”, aerosol bombardment devices, or other physical methods such as electroporation (“EP”), “hydrodynamic method”, or ultrasound. Electroporation apparatuses capable of performing the in vivo electroporation for transfecting in situ cells with one or more of the powassan constructs described herein include any commercially available electroporation devices. In addition, other publicly describe electroporation devices may be utilized upon configuring the device to perform the desired electroporation to cause reversible member pores to form and maintain the viability of the cells. The electroporation devices can include such devices described and made or utilized by the following companies: Inovio Pharmaceuticals (Cellectra), Cellectis (Easy Vax), Ichor (Trigrid), IGEA (CliniPorator), BTX Harvard, among others.

The plasmid of the immunogenic composition may be delivered to the mammal by several well-known technologies including DNA or RNA injection (also referred to as nucleic acid vaccination) with and without in vivo electroporation, liposome mediated, nanoparticle facilitated, recombinant vectors such as recombinant adenovirus, recombinant adenovirus associated virus and recombinant vaccinia.

### Generation of Antigens In Vitro and Ex Vivo

In one embodiment, the optimized consensus POWV antigen is generated in vitro or ex vivo. For example, in one embodiment, a nucleic acid encoding an optimized consensus POWV antigen can be introduced and expressed in an in vitro or ex vivo cell.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). Preferred methods for the introduction of a polynucleotide into a host cell are calcium phosphate transfection, lipid nanoparticles, and silica nanoparticles.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1 Origin of Plasmid Backbone

pVAX1 (Invitrogen V260-20) was purchased from Thermo Fisher Scientific and a master cell bank was established in Escherichia coli strain DH10B-TIR. The purified plasmid DNA derived from the master cell bank was delivered to GenScript for constructing five powassan virus (POWV) DNA vaccines described, below.

### Example 2 Construction of Powassan virus (POWV) DNA Vaccines

Five POWV DNA candidate vaccines were designed and constructed with the same antigen sequence, which encodes the POWV pre-membranemembrane, and envelope (prME) genes with different signal peptide sequences and with and without leader sequence.

The consensus prME protein coding sequence was generated from 22 different POWV prM protein sequences and 37 different POWV E protein sequences using MegAlign software (DNASTAR, Madison, WI). The signal peptide sequence of the five constructs contained either (1) an IgE leader sequence alone, (2) an IgE leader sequence plus the 20 amino acid consensus prM signal sequence, (3) an IgE leader plus the first 12 amino acids of the prM signal sequence, (4) the 20 amino acid consensus prM signal sequence, and (5) a JEV signal sequence, respectively. The prM signal sequence was generated as a consensus sequence from 22 different POWV prM signal peptide sequences.

The Genbank accession numbers used for generating the consensus envelope (E) sequences are: AAL32141.1, AAL32142. 1, AAL32143.1, AAL32144.1, AAL32145.1, AAL32146.1, AAL32147.1, AAL32148.1, AAL32149.1, AAL32150.1, AAL32151.1, AAL32152. 1, AAL32153.1, AAL32154.1, AAL32169.1, AAK61347.1, ACD67873.1, ACD88752.1, ACF05267.1, ADK37752.1, ADK37753.1, ADK37754.1, ADK37755.1, ADK37756.1, ADK37757.1, AEN83681.1, AEN83682.1, AIG95651.1, ALP82430.1, ALP82431.1, AAA02739.1, AMY50382.1, AMZ80134.1, AMQ49162.1, AMQ49164.1, AMQ49165.1, AMQ49163.1

The Genbank accession numbers used for generating the consensus pre-membrane (prM) and prM signal sequences are: AAL32169.1, AAK61347.1, ACD67873.1, ACD88752.1, ACF05267.1, ADK37752.1, ADK37753.1, ADK37754.1, ADK37755.1, ADK37756.1, ADK37757.1, AEN83681.1, AEN83682.1, AIG95651.1, ALP82430.1, ALP82431.1, AAA02739.1, AMY50382.1, AMQ49162.1, AMQ49164.1, AMQ49165.1, AMQ49163.1

Once the consensus prME sequence was obtained, each signal peptide sequence (with and without leader sequence) of the five constructs was added to the N-terminal to facilitate expression and mRNA export, and further modified with upstream Kozak sequence. This sequence was then codon optimized and synthesized by GenScript. The synthesized insert was subcloned into the pVAX1 at the BamHI and XhoI sites, creating constructs:
Candidate 1 pVAX1-IgE-prME (SEQ ID NO:1)
Candidate 2 pVAX1-IgE-prMsignal-prME (SEQ ID NO:2)
Candidate 3 pVAX1-IgE-portion of prMsignal-prME (SEQ ID NO:3)
Candidate 4 pVAX1-prMsignal-prME (SEQ ID NO:4)
Candidate 5 pVAX1-JEVsignal-prME (SEQ ID NO:5)

### Example 3 Detection of Expressed POWV prM Protein in Transfected Cells

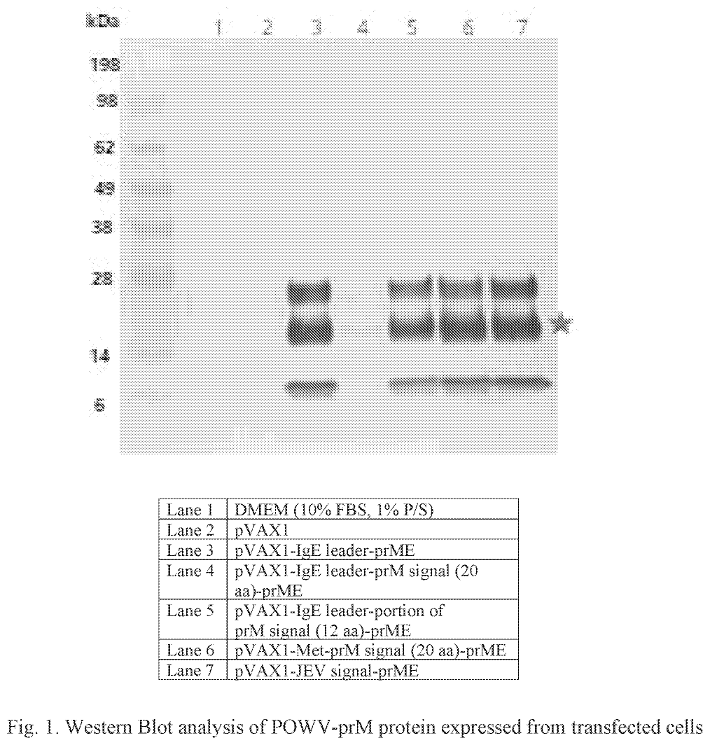

To assess expression of the vaccine candidates, HEK293T cells were transfected with POWV DNA vaccines, respectively, using X-fect reagent (Clontech, Takara Korea Biomedical, Inc., Seoul Korea) and the cell supernatants were collected 48 hours after transfection. Total protein (20 μg) was subjected to a SDS-PAGE under reducing condition and analyzed by western blot using POWV prM polyclonal antibody (GeneTex Inc., Irvine, CA). The Western blot data indicated that four of the five POWV DNA vaccines expressed POWV-prM protein having approx. 18 KDa. One vaccine candidate containing two complete signal sequences, the IgE-leader sequence plus prM signal sequence, did not show evidence of prM expression (FIG. 1.).

### Example 4. Induction of POWV Specific Binding Antibody in Mice by the POWV DNA Vaccination

POWV prME antigen specific binding antibody in sera from immunized C57BL/6 mice was measured by ELISA using recombinant POWV-E (rPOWV-E) (Bioclone Inc., Sandiego, CA) as a coating antigen and the ability of the POWV vaccines to induce antibody immune response was evaluated between groups. Mice in each group were immunized with 25 μg of POWV DNA vaccine by intramuscular (i.m.) electroporation for 3 times 2 weeks apart. The sera were collected at day 0 (1 day before the first injection), day 21 (1 week after the second injection), day 35 (1 week after the third injection), and day 60 (approx. 4 weeks after the third injection).

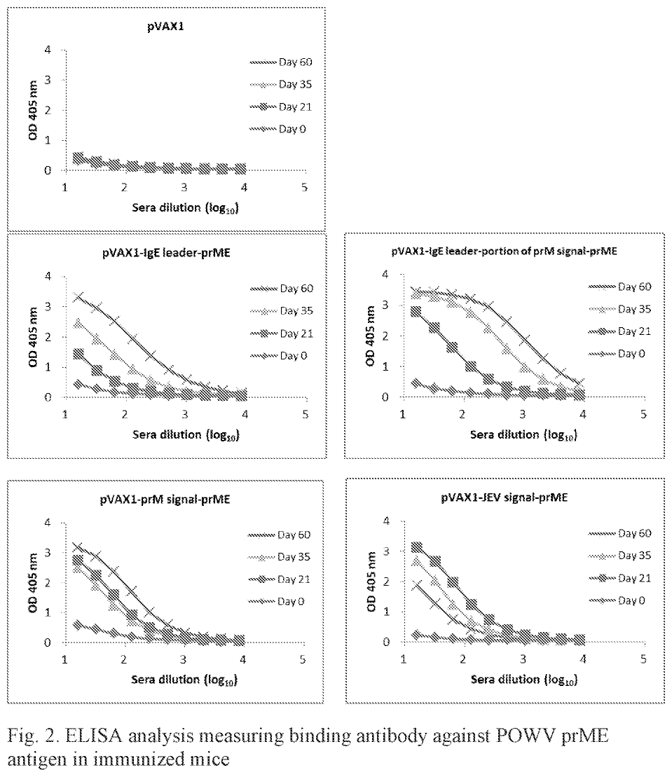

POWV specific antibody levels were significantly elevated in all groups on day 21, 1 week after the second immunization, except pVAX1 control group. However, in pVAX1-JEV signal-prME group, POWV specific humoral responses were not further boosted by third immunization (FIG. 2.).

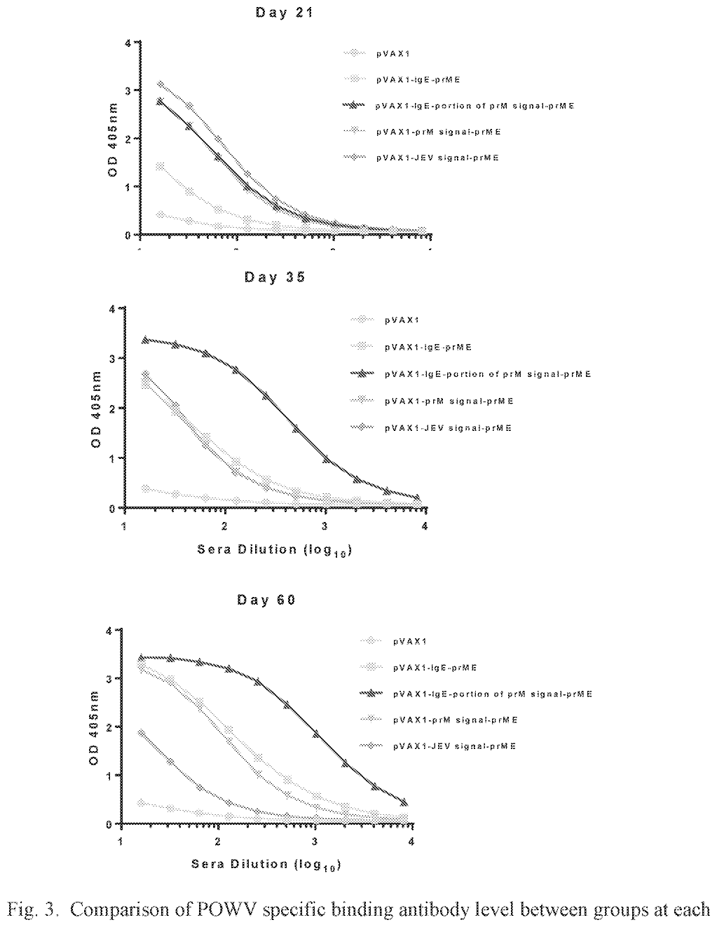

The data sorted by timepoint was shown that binding antibody against rPOWV-E was markedly increased in pVAX1-IgE leader-portion of prM signal-prME group on day 35, 1 week after the third immunization, and maintained for 4 more weeks thereafter. This result suggests that POWV vaccine having the IgE leader and the portion of prM signal sequence together as signal peptide sequence is more potent than other groups in inducing POWV-E specific binding antibodies (FIG. 3.).

The endpoint titer was determined as the highest dilution of sera meeting the cut-off value which is absorbance above four times the mean of naïve (preimmunization) and 0.15. The cut-off value applied in this study is more stringent than the ‘mean +2 or 3 SD’ or the ‘2 or 3 mean’ reading that generally used in ELISA.

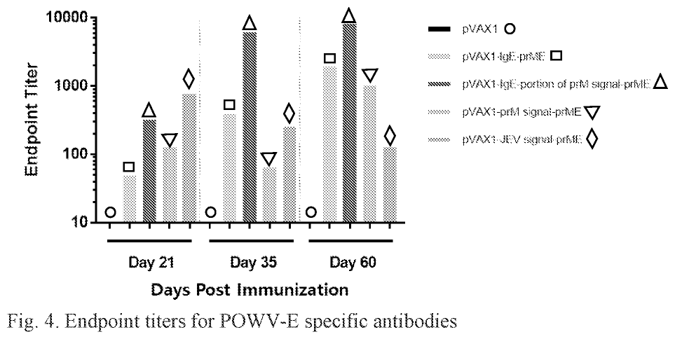

As shown in FIG. 4., the group immunized with POWV vaccine having IgE leader-portion of prM signal sequence was distinguished from other groups in terms of high endpoint titers on day 35 and 60. And interestingly, two groups, pVAX1-IgE leader-prME and pVAX1-IgE leader-portion of prM signal-prME, represented time dependent increase of humoral responses and the high endpoint titers was on day 60 (FIG. 4.)

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. An immunological composition comprising a nucleotide sequence encoding a consensus POWV antigen, wherein the consensus POWV antigen comprises an amino acid sequence selected from the group consisting of SEQ ID NO:2, SEQ ID NO:4, an amino acid sequence that is 90% identical or greater to SEQ ID NO:2, and an amino acid sequence that is 90% identical or greater to SEQ ID NO:4.

2. The immunological composition of claim 1, wherein the nucleotide sequence comprises a nucleotide sequence selected from the group consisting of: SEQ ID NO:1, SEQ ID NO:3, a nucleotide sequence that is 90% identical or greater to SEQ ID NO:1, and a nucleotide sequence that is 90% identical or greater to SEQ ID NO:3.

3. The immunogenic composition of claim 1, wherein the nucleic acid molecule is selected from the group consisting of a DNA molecule and an RNA molecule.

4. The immunological composition of claim 1, wherein the nucleotide sequence comprises one or more plasmids.

5. The immunological composition of claim 1, further comprising a nucleotide sequence encoding an adjuvant.

6. The immunogenic composition of claim 1, wherein a nucleotide sequence encoding the peptide is operably linked to at least one regulatory sequence selected from the group consisting of a start codon, an IgE leader sequence and a stop codon.

7. The immunogenic composition of claim 1, wherein the nucleic acid molecule comprises an expression vector.

8. The immunogenic composition of claim 1, wherein the nucleic acid molecule is incorporated into a viral particle.

9. The immunogenic composition of claim 1, further comprising a pharmaceutically acceptable excipient.

10. A method of treating or preventing POWV infection in a subject in need thereof, the method comprising administering the immunological composition of claim 1 to the subject.

11. The method of claim 12, wherein the administering step comprises electroporation, aerosol administration, gene-gun administration, microneedle injection, or syringe needle injection.

12. A method of inducing an immune response to POWV in a subject in need thereof, the method comprising administering an immunogenic composition of claim 1 to the subject.

13. The method of claim 12, wherein the administering step comprises electroporation.

14. A nucleic acid molecule comprising one or more nucleotide sequences selected from the group consisting of: SEQ ID NO:1, SEQ ID NO:3, a nucleotide sequence that is 90% identical or greater to SEQ ID NO:1, and a nucleotide sequence that is 90% identical or greater to SEQ ID NO:3.

15. A protein comprising one or more amino acid sequences selected from the group consisting of: SEQ ID NO:2, SEQ ID NO:4, an amino acid sequence that is 90% identical or greater to SEQ ID NO:2, and an amino acid sequence that is 90% identical or greater to SEQ ID NO:4.


SEQUENCE LISTING (9 sequences)

SEQ ID NO: 1 - DNA, 4997 bp, Artificial Sequence
  pVAX1-IgE-prME
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggcagcagcaacaagggtgcacagcaccacaatccacagggaccgcgagggctacatggtcatgagggcaagcggaagagatgcagcatcccaggtgcgcgtgcagaacggaacctgcgtgatcctggccacagacatgggcgagtggtgcgaggattccatcacctactcttgcgtgacaatcgaccaggaggaggagcctgtggacgtggattgcttttgtaggggcgtggatcgcgtgaagctggagtatggccggtgtggcagacaggcaggcagccggggcaagcggagcgtggtcatcccaacccacgcacagaaggacatggtgggaaggggacacgcatggctgaagggcgacaacatcagggatcacgtgacccgcgtggagggctggatgtggaagaataagctgctgacagcagcaatcgtggccctggcatggctgatggtggactcttggatggcccgggtgaccgtgatcctgctggccctgagcctgggacccgtgtacgcaaccaggtgcacacacctggagaaccgcgatttcgtgaccggcacacagggcaccacaagagtgtccctggtgctggagctgggaggatgcgtgaccatcacagcagagggcaagccaagcatcgacgtgtggctggaggatatctttcaggagtccccagcagagacccgggagtattgcctgcacgccaagctgaccaatacaaaggtggaggccagatgtcccaccacaggccctgccacactgccagaggagcaccaggccaacatggtgtgcaagcgggaccagtccgatagaggctggggcaatcactgtggcttctttggcaagggctctatcgtggcctgcgccaagttcgagtgtgaggaggccaagaaggccgtgggccacgtgtacgactctaccaagatcacatatgtggtgaaggtggagccacacaccggcgattacctggcagccaacgagacaaacagcaataggaagtccgcccagtttaccgtggccagcgagaaagtgatcctgcgcctgggcgactatggcgacgtgagcctgacctgcaaggtggcaagcggaatcgacgtggcacagacagtggtcatgtctctgggcagctccaaggatcacctgcctagcgcctggcaggtgcacagggactggttcgaggatctggccctgccatggaagcacaaggacaaccaggattggaattccgtggagaagctggtggagttcggaccacctcacgcagtgaagatggacgtgtttaatctgggcgatcagaccgccgtgctgctgaagtccctggcaggagtgcctctggcatctgtggagggccagaagtaccacctgaagtctggacacgtgacctgcgacgtgggactggagaagctgaagctgaagggcaccacatatagcatgtgcgataaggccaagtttaagtggaagagggtgccagtggactccggacacgataccgtggtcatggaggtgtcttacacaggcagcgacaagccatgtagaatccccgtgcgggcagtggcacacggagtgcccgccgtgaacgtggccatgctgatcacccctaacccaaccatcgagacaaatggcggcggcttcatcgagatgcagctgccacccggcgacaatatcatctatgtgggcgatctgagccagcagtggttccagaagggctccaccatcggccggatgtttgagaagacacggagaggcctggagagactgtccgtggtgggagagcacgcatgggattttggctctgtgggaggcgtgctgtctagcgtgggcaaggcaatccacaccgtgctgggaggcgccttcaatacactgtttggcggcgtgggcttcatccccaagatgctgctgggagtggccctggtgtggctgggcctgaacgcccggaatcctaccatgagcatgacatttctggccgtgggcgccctgaccctgatgatgacaatgggcgtgggcgcctaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 2 - DNA, 5060 bp, Artificial Sequence
  pVAX1-IgE-prM signal-prME
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggcagcagcaacacgggtgcacagcatgagcggcgtggactggacctggatcttcctgacaatggccctgatgaccatggccatggccaccacaatccacagggaccgcgagggctacatggtcatgagggcaagcggaagagatgcagcatcccaggtgcgggtgcagaacggaacatgcgtgatcctggccaccgacatgggcgagtggtgcgaggattccatcacatactcttgcgtgaccatcgaccaggaggaggagcctgtggacgtggattgcttttgtaggggcgtggatcgcgtgaagctggagtatggccggtgtggcagacaggcaggcagccggggcaagcggagcgtggtcatcccaacccacgcccagaaggacatggtgggaagaggacacgcatggctgaagggcgacaacatcagggatcacgtgacacgcgtggagggctggatgtggaagaataagctgctgaccgcagcaatcgtggccctggcctggctgatggtggactcttggatggccagggtgaccgtgatcctgctggccctgagcctgggacccgtgtacgcaaccaggtgcacacacctggagaaccgcgatttcgtgaccggaacacagggaaccacacgcgtgtccctggtgctggagctgggaggatgcgtgaccatcacagcagagggcaagccaagcatcgacgtgtggctggaggatatctttcaggagtcccccgccgagacacgggagtattgcctgcacgccaagctgaccaatacaaaggtggaggccagatgtccaaccacaggacctgccaccctgccagaggagcaccaggccaacatggtgtgcaagcgggaccagtccgatagaggctggggcaatcactgtggcttctttggcaagggctctatcgtggcctgcgccaagttcgagtgtgaggaggccaagaaggccgtgggccacgtgtacgactctaccaagatcacatatgtggtgaaggtggagccccacacaggcgattacctggccgccaacgagaccaacagcaataggaagtccgcccagtttaccgtggccagcgagaaagtgatcctgcgcctgggcgactatggcgacgtgagcctgacatgcaaggtggcaagcggaatcgacgtggcacagaccgtggtcatgtctctgggcagctccaaggatcacctgcctagcgcctggcaggtgcacagggactggttcgaggatctggccctgccatggaagcacaaggacaaccaggattggaattccgtggagaagctggtggagttcggaccacctcacgcagtgaagatggacgtgtttaatctgggcgatcagacagccgtgctgctgaagtccctggcaggagtgcctctggcatctgtggagggccagaagtaccacctgaagtctggacacgtgacctgcgacgtgggactggagaagctgaagctgaagggcaccacatatagcatgtgcgataaggccaagtttaagtggaagagggtgccagtggactccggacacgatacagtggtcatggaggtgtcttacaccggcagcgacaagccatgtagaatccccgtgcgggcagtggcacacggagtgcccgccgtgaacgtggccatgctgatcacccctaacccaaccatcgagacaaatggcggcggcttcatcgagatgcagctgccacccggcgacaatatcatctatgtgggcgatctgagccagcagtggttccagaagggctccacaatcggccggatgtttgagaagacccggagaggcctggagagactgtccgtggtgggagagcacgcatgggattttggctctgtgggaggcgtgctgtctagcgtgggcaaggccatccacacagtgctgggcggcgccttcaataccctgtttggcggcgtgggcttcatccccaagatgctgctgggagtggccctggtgtggctgggcctgaacgcaaggaatcctaccatgagcatgacatttctggccgtgggcgccctgaccctgatgatgacaatgggcgtgggcgcctaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 3 - DNA, 5033 bp, Artificial Sequence
  pVAX1-IgE-portion of prM signal-prME
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggcagcagcaacacgggtgcacagctttctgaccatggccctgatgacaatggccatggccaccacaatccacagggaccgcgagggctacatggtcatgagggcaagcggaagagatgcagcatcccaggtgagagtgcagaacggcacctgcgtgatcctggccacagacatgggcgagtggtgcgaggattccatcacctactcttgcgtgacaatcgaccaggaggaggagcctgtggacgtggattgcttctgtaggggcgtggatcgcgtgaagctggagtatggccggtgtggcagacaggcaggcagccggggcaagcggagcgtggtcatcccaacccacgcacagaaggacatggtgggaaggggacacgcatggctgaagggcgacaacatcagggatcacgtgacccgcgtggagggctggatgtggaagaataagctgctgacagcagcaatcgtggccctggcatggctgatggtggactcttggatggccagggtgaccgtgatcctgctggccctgagcctgggacccgtgtacgcaaccaggtgcacacacctggagaaccgcgatttcgtgaccggaacacagggaaccacacgcgtgtccctggtgctggagctgggaggatgcgtgaccatcacagcagagggcaagccaagcatcgacgtgtggctggaggatatctttcaggagtccccagcagagacccgggagtattgcctgcacgccaagctgaccaatacaaaggtggaggccagatgtcccaccacaggccctgccacactgccagaggagcaccaggccaacatggtgtgcaagcgggaccagtccgatagaggctggggcaatcactgtggcttctttggcaagggctctatcgtggcctgcgccaagttcgagtgtgaggaggccaagaaggccgtgggccacgtgtacgactctaccaagatcacatatgtggtgaaggtggagccacacaccggcgattacctggcagccaacgagacaaacagcaataggaagtccgcccagtttaccgtggccagcgagaaagtgatcctgcgcctgggcgactatggcgacgtgagcctgacctgcaaggtggcaagcggaatcgacgtggcacagacagtggtcatgtctctgggcagctccaaggatcacctgcctagcgcctggcaggtgcacagggactggttcgaggatctggccctgccatggaagcacaaggacaaccaggattggaattccgtggagaagctggtggagttcggaccacctcacgcagtgaagatggacgtgtttaatctgggcgatcagaccgccgtgctgctgaagtccctggcaggagtgcctctggcatctgtggagggccagaagtaccacctgaagtctggacacgtgacctgcgacgtgggactggagaagctgaagctgaagggcaccacatatagcatgtgcgataaggccaagtttaagtggaagagggtgccagtggactccggacacgataccgtggtcatggaggtgtcttacacaggcagcgacaagccatgtagaatccccgtgcgggcagtggcacacggagtgcccgccgtgaacgtggccatgctgatcacccctaacccaaccatcgagacaaatggcggcggcttcatcgagatgcagctgccacccggcgacaatatcatctatgtgggcgatctgagccagcagtggttccagaagggctccaccatcggccggatgtttgagaagacacggagaggcctggagagactgtccgtggtgggagagcacgcatgggattttggctctgtgggaggcgtgctgtctagcgtgggcaaggcaatccacaccgtgctgggaggcgccttcaatacactgtttggcggcgtgggcttcatccccaagatgctgctgggagtggccctggtgtggctgggcctgaacgcccggaatcctaccatgagcatgacatttctggccgtgggcgccctgaccctgatgatgacaatgggcgtgggcgcctaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 4 - DNA, 5006 bp, Artificial Sequence
  pVAX1-prM signal-prME
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatgagcggcgtggactggacctggatcttcctgacaatggccctgatgaccatggccatggccaccacaatccacagggaccgcgagggctacatggtcatgagggcaagcggaagagatgcagcatcccaggtgcgggtgcagaacggaacatgcgtgatcctggccaccgacatgggcgagtggtgcgaggattccatcacatactcttgcgtgaccatcgaccaggaggaggagcctgtggacgtggattgcttttgtaggggcgtggatcgcgtgaagctggagtatggccggtgtggcagacaggcaggcagccggggcaagcggagcgtggtcatcccaacccacgcccagaaggacatggtgggaagaggacacgcatggctgaagggcgacaacatcagggatcacgtgacacgcgtggagggctggatgtggaagaataagctgctgaccgcagcaatcgtggccctggcctggctgatggtggactcttggatggccagggtgaccgtgatcctgctggccctgagcctgggacccgtgtacgcaaccaggtgcacacacctggagaaccgcgatttcgtgaccggaacacagggaaccacacgcgtgtccctggtgctggagctgggaggatgcgtgaccatcacagcagagggcaagccaagcatcgacgtgtggctggaggatatctttcaggagtcccccgccgagacacgggagtattgcctgcacgccaagctgaccaatacaaaggtggaggccagatgtccaaccacaggacctgccaccctgccagaggagcaccaggccaacatggtgtgcaagcgggaccagtccgatagaggctggggcaatcactgtggcttctttggcaagggctctatcgtggcctgcgccaagttcgagtgtgaggaggccaagaaggccgtgggccacgtgtacgactctaccaagatcacatatgtggtgaaggtggagccccacacaggcgattacctggccgccaacgagaccaacagcaataggaagtccgcccagtttaccgtggccagcgagaaagtgatcctgcgcctgggcgactatggcgacgtgagcctgacatgcaaggtggcaagcggaatcgacgtggcacagaccgtggtcatgtctctgggcagctccaaggatcacctgcctagcgcctggcaggtgcacagggactggttcgaggatctggccctgccatggaagcacaaggacaaccaggattggaattccgtggagaagctggtggagttcggaccacctcacgcagtgaagatggacgtgtttaatctgggcgatcagacagccgtgctgctgaagtccctggcaggagtgcctctggcatctgtggagggccagaagtaccacctgaagtctggacacgtgacctgcgacgtgggactggagaagctgaagctgaagggcaccacatatagcatgtgcgataaggccaagtttaagtggaagagggtgccagtggactccggacacgatacagtggtcatggaggtgtcttacaccggcagcgacaagccatgtagaatccccgtgcgggcagtggcacacggagtgcccgccgtgaacgtggccatgctgatcacccctaacccaaccatcgagacaaatggcggcggcttcatcgagatgcagctgccacccggcgacaatatcatctatgtgggcgatctgagccagcagtggttccagaagggctccacaatcggccggatgtttgagaagacccggagaggcctggagagactgtccgtggtgggagagcacgcatgggattttggctctgtgggaggcgtgctgtctagcgtgggcaaggccatccacacagtgctgggcggcgccttcaataccctgtttggcggcgtgggcttcatccccaagatgctgctgggagtggccctggtgtggctgggcctgaacgcaaggaatcctaccatgagcatgacatttctggccgtgggcgccctgaccctgatgatgacaatgggcgtgggcgcctaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 5 - DNA, 5015 bp, Artificial Sequence
  pVAX1-JEV signal-prME
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatgggcaagcggtccgccggctctatcatgtggctggccagcctggcagtggtcatcgcatgcgcaggagcaaccacaatccacagggaccgcgagggctacatggtcatgagggcatctggaagagatgcagcaagccaggtgagagtgcagaacggcacctgcgtgatcctggccacagacatgggcgagtggtgcgaggatagcatcacctactcctgcgtgacaatcgaccaggaggaggagcctgtggacgtggattgcttctgtaggggcgtggatcgcgtgaagctggagtatggccggtgcggcagacaggcaggcagccggggcaagcggagcgtggtcatcccaacccacgcacagaaggacatggtgggaaggggacacgcatggctgaagggcgacaacatcagggatcacgtgacccgcgtggagggctggatgtggaagaataagctgctgacagcagcaatcgtggccctggcatggctgatggtggactcctggatggccagggtgaccgtgatcctgctggccctgtctctgggacccgtgtacgcaaccaggtgcacacacctggagaaccgcgatttcgtgaccggaacacagggaaccacacgcgtgtccctggtgctggagctgggaggatgcgtgaccatcacagcagagggcaagccatctatcgacgtgtggctggaggatatctttcaggagagcccagcagagacccgggagtattgcctgcacgccaagctgaccaatacaaaggtggaggccagatgtcccaccacaggccctgccacactgccagaggagcaccaggccaacatggtgtgcaagcgggaccagagcgatagaggctggggcaatcactgtggcttctttggcaagggctccatcgtggcctgcgccaagttcgagtgtgaggaggccaagaaggccgtgggccacgtgtacgactccaccaagatcacatatgtggtgaaggtggagccacacaccggcgattacctggcagccaacgagacaaactctaataggaagagcgcccagtttaccgtggcctctgagaaagtgatcctgcgcctgggcgactatggcgacgtgagcctgacctgcaaggtggcctctggcatcgacgtggcccagacagtggtcatgtccctgggcagctccaaggatcacctgccttctgcctggcaggtgcacagggactggttcgaggatctggccctgccatggaagcacaaggacaaccaggattggaatagcgtggagaagctggtggagttcggaccacctcacgcagtgaagatggacgtgtttaatctgggcgatcagaccgccgtgctgctgaagagcctggcaggagtgcctctggcatccgtggagggccagaagtaccacctgaagtccggacacgtgacctgcgacgtgggactggagaagctgaagctgaagggcaccacatattctatgtgcgataaggccaagtttaagtggaagagggtgccagtggacagcggacacgataccgtggtcatggaggtgtcctacacaggctctgacaagccatgtagaatccccgtgcgggcagtggcacacggagtgcccgccgtgaacgtggccatgctgatcacccctaacccaaccatcgagacaaatggcggcggcttcatcgagatgcagctgccacccggcgacaatatcatctatgtgggcgatctgtctcagcagtggttccagaagggcagcaccatcggccggatgtttgagaagacacggagaggcctggagagactgagcgtggtgggagagcacgcatgggattttggctccgtgggaggcgtgctgtctagcgtgggcaaggcaatccacaccgtgctgggaggcgccttcaatacactgtttggcggcgtgggcttcatccccaagatgctgctgggagtggccctggtgtggctgggcctgaacgcccggaatcctaccatgagcatgacatttctggccgtgggcgccctgaccctgatgatgacaatgggcgtgggcgcctaatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 6 - DNA, 54 bp, Artificial Sequence
  IgE leader
atggactggacctggattctgttcctggtggcagcagcaacacgggtgcacagc

SEQ ID NO: 7 - protein, 164 aa, Artificial Sequence
  PrM consensus sequence
TTIHRDREGYMVMRASGRDAASQVRVQNGTCVILATDMGEWCEDSITYSCVTIDQEEEPVDVDCFCRGVDRVKLEYGRCGRQAGSRGKRSVVIPTHAQKDMVGRGHAWLKGDNIRDHVTRVEGWMWKNKLLTAAIVALAWLMVDSWMARVTVILLALSLGPVYA

SEQ ID NO: 8 - protein, 497 aa, Artificial Sequence
  E consensus sequence
  misc_feature (430)..(430): Xaa can be any naturally occurring amino acid
TRCTHLENRDFVTGTQGTTRVSLVLELGGCVTITAEGKPSIDVWLEDIFQESPAETREYCLHAKLTNTKVEARCPTTGPATLPEEHQANMVCKRDQSDRGWGNHCGFFGKGSIVACAKFECEEAKKAVGHVYDSTKITYVVKVEPHTGDYLAANETNSNRKSAQFTVASEKVILRLGDYGDVSLTCKVASGIDVAQTVVMSLGSSKDHLPSAWQVHRDWFEDLALPWKHKDNQDWNSVEKLVEFGPPHAVKMDVFNLGDQTAVLLKSLAGVPLASVEGQKYHLKSGHVTCDVGLEKLKLKGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQKGSTIGRMFEKTRRGLERLSVVGEHAWDFGSVGGXLSSVGKAIHTVLGGAFNTLFGGVGFIPKMLLGVALVWLGLNARNPTMSMTFLAVGALTLMMTMGVGA

SEQ ID NO: 9 - DNA, 1989 bp, Artificial Sequence
  prME consensus
accacaatccacagggaccgcgagggctacatggtcatgagggcaagcggaagagatgcagcatcccaggtgcgcgtgcagaacggaacctgcgtgatcctggccacagacatgggcgagtggtgcgaggattccatcacctactcttgcgtgacaatcgaccaggaggaggagcctgtggacgtggattgcttttgtaggggcgtggatcgcgtgaagctggagtatggccggtgtggcagacaggcaggcagccggggcaagcggagcgtggtcatcccaacccacgcacagaaggacatggtgggaaggggacacgcatggctgaagggcgacaacatcagggatcacgtgacccgcgtggagggctggatgtggaagaataagctgctgacagcagcaatcgtggccctggcatggctgatggtggactcttggatggcccgggtgaccgtgatcctgctggccctgagcctgggacccgtgtacgcaaccaggtgcacacacctggagaaccgcgatttcgtgaccggcacacagggcaccacaagagtgtccctggtgctggagctgggaggatgcgtgaccatcacagcagagggcaagccaagcatcgacgtgtggctggaggatatctttcaggagtccccagcagagacccgggagtattgcctgcacgccaagctgaccaatacaaaggtggaggccagatgtcccaccacaggccctgccacactgccagaggagcaccaggccaacatggtgtgcaagcgggaccagtccgatagaggctggggcaatcactgtggcttctttggcaagggctctatcgtggcctgcgccaagttcgagtgtgaggaggccaagaaggccgtgggccacgtgtacgactctaccaagatcacatatgtggtgaaggtggagccacacaccggcgattacctggcagccaacgagacaaacagcaataggaagtccgcccagtttaccgtggccagcgagaaagtgatcctgcgcctgggcgactatggcgacgtgagcctgacctgcaaggtggcaagcggaatcgacgtggcacagacagtggtcatgtctctgggcagctccaaggatcacctgcctagcgcctggcaggtgcacagggactggttcgaggatctggccctgccatggaagcacaaggacaaccaggattggaattccgtggagaagctggtggagttcggaccacctcacgcagtgaagatggacgtgtttaatctgggcgatcagaccgccgtgctgctgaagtccctggcaggagtgcctctggcatctgtggagggccagaagtaccacctgaagtctggacacgtgacctgcgacgtgggactggagaagctgaagctgaagggcaccacatatagcatgtgcgataaggccaagtttaagtggaagagggtgccagtggactccggacacgataccgtggtcatggaggtgtcttacacaggcagcgacaagccatgtagaatccccgtgcgggcagtggcacacggagtgcccgccgtgaacgtggccatgctgatcacccctaacccaaccatcgagacaaatggcggcggcttcatcgagatgcagctgccacccggcgacaatatcatctatgtgggcgatctgagccagcagtggttccagaagggctccaccatcggccggatgtttgagaagacacggagaggcctggagagactgtccgtggtgggagagcacgcatgggattttggctctgtgggaggcgtgctgtctagcgtgggcaaggcaatccacaccgtgctgggaggcgccttcaatacactgtttggcggcgtgggcttcatccccaagatgctgctgggagtggccctggtgtggctgggcctgaacgcccggaatcctaccatgagcatgacatttctggccgtgggcgccctgaccctgatgatgacaatgggcgtgggcgcctaatga